(19) United States
(12) Patent Application Publication
(10) Pub. No.: US 2014/0135786 A1
(43) Pub. Date: May 15, 2014

(54) MEDICAL PROCEDURE ACCESS KIT

(71) Applicant: NARIS LLC, Carmel, IN (US)

(72) Inventor: Saihari Sadanandan, Carmel, IN (US)

(73) Assignee: NARIS LLC, Carmel, IN (US)

(21) Appl. No.: 13/673,976

(22) Filed: Nov. 9, 2012

(51) Int. Cl. A61B 18/00

(52) U.S. Cl. CPC A61B 18/00
USPC 606/108

(57) ABSTRACT

A medical procedure access kit for inserting a medical device into a biological tubular structure includes at least one semi-flexible sheath and at least one semi-flexible angled guidewire. The sheath defines an internal side wall and an external side wall. The sheath includes a longitudinal opening defined by the internal side wall, a pre-formed bend along a length of the sheath, and a side hole disposed on the pre-formed bend and extending from the external side wall to the internal side wall. The angled guidewire is sized and configured so that it can be received within the sheath.

### FIELD

This application relates to the field of medical procedure introducers and, particularly, to introducers capable of insertion into and removal from a biological tubular structure in a retrograde direction.

### BACKGROUND

Introducer sheaths are used for inserting catheters, guidewires, leads, stents, embolic protection devices, implants, and other medical devices into a biological tubular structure of a patient. One common example, used herein for illustrative purposes, is using an introducer sheath to insert a catheter into a patient's vessel. In one common practice, a physician inserts the introducer sheath into the patient using the Seldinger Technique. In this technique, the patient's vascular system is accessed by puncturing a vessel with a needle. Next, once the vessel bleeds back into the needle, indicating that the vascular system has been accessed, the physician inserts a guidewire through the needle and into the vascular system. The physician removes the needle, leaving the guidewire in the vessel. The physician then places an introducer sheath over the guidewire and inserts the sheath into the vessel to provide a working tunnel or port for devices. The introducer sheath provides access between the inside of the patient's vessel and the outside of the patient's body. While the example of a patient's vascular system is used herein for illustrative purposes, it is understood that the description below also applies to other biological tubular structures that are found outside of the vascular system. For example, the following disclosure can also apply to bronchial tubes in a patient's respiratory system or to any other biological tubular structure within the patient.

Examples of introducer sheaths are disclosed in U.S. Pat. No. 7,909,798 to Osypka, U.S. Pat. No. 5,304,156 to Sylvanowicz, U.S. Pat. No. 5,779,681 to Bonn, and U.S. Pat. No. 7,204,831 to McGuckin, Jr., the entireties of which are incorporated herein by reference.

When an introducer sheath is positioned within a patient's vessel, it is either positioned in the antegrade direction, with the flow of blood (or other fluid), or in the retrograde direction, against the flow of blood (or other fluid). Generally, once the physician inserts the introducer sheath into a vessel in a particular direction, the physician carries out work within the vessel in that same direction. In other words, if work is done “upstream” of the access point in the vessel, then the introducer sheath is inserted in the retrograde direction. If work is done “downstream” of the access point, then the sheath is inserted in the antegrade direction. However, there are advantages and disadvantages associated with inserting and exiting the patient's vessel in either of the directions.

One factor that physicians might consider when determining how to insert an introducer sheath into a patient's vessel is the relative ease and effectiveness with which the introducer sheath can be inserted in the retrograde direction. By contrast, inserting the introducer sheath into a vessel in the antegrade direction can be more complex and more likely to result in vascular access complications. The ease and efficacy of entry into a vessel tends to cause physicians to prefer performing a procedure in a patient's vessel in the retrograde direction.

An additional factor that physicians might consider when determining how to insert an introducer sheath into a patient's vessel is the subsequent closing procedure, and more specifically, the removal of the introducer sheath from the patient's vessel. There are currently no vascular access closure devices specifically dedicated to closing procedures conducted in the antegrade direction. Accordingly, ease and efficacy of closure of a vessel is another factor that tends to cause physicians to favor performing a procedure in a patient's vessel in the retrograde direction.

To address the shortcomings of inserting and removing introducer sheaths in the antegrade direction, some techniques have been developed to facilitate working in the antegrade direction using a retrograde-inserted sheath. By way of example, when working in the femoral artery, entry into the femoral artery is typically conducted in the retrograde direction for the reasons discussed above. Many procedures, however, are typically conducted in the antegrade direction.

In order to take advantage of the retrograde access and still facilitate procedures at points antegrade (or downstream) of the point of entry, one prior art practice involves obtaining retrograde access to the femoral artery in one leg to obtain antegrade access to blood vessels in the contralateral leg. This method of access is referred to as contralateral retrograde access. In particular, for example, in order to reach the superficial femoral artery (hereinafter “SFA”) on the left side of a patient's body, retrograde entry is typically made at the common femoral artery (hereinafter “CFA”) on the right side of the patient's body. The medical procedure equipment is then fed into the left SFA in the retrograde direction, upstream through the right external iliac artery (hereinafter “EIA”) to the right common iliac artery (hereinafter “CIA”), and then back downstream through the left CIA and the left EIA into the left SFA in the antegrade direction. (Although contralateral retrograde access is most common, this procedure requires a large amount of transmission within the arteries, requiring fine skill in certain situations and has its own limitations.

Additionally, performing procedures using this contralateral retrograde prior art practice introduces countervailing disadvantages. One factor that physicians can consider when determining whether to insert an introducer sheath into a patient's vessel in this manner is the length of the equipment relative to the location of the procedure to be performed on the patient. Inserting procedure equipment in the retrograde direction near the patient's right hip and feeding the equipment through the vessels and then into the antegrade direction in the left side of the patient's body can be problematic, for example, in tall patients. Because equipment for this type of medical procedure is typically a standard length, for example, 135 cm, the equipment might not be long enough to reach the patient's left ankle when inserted in the retrograde direction at the right hip. Accordingly, the potential for standard length equipment to be too short relative to the location of the procedure to be performed on a patient is a factor that tends to cause a physician to prefer performing a procedure in a patient's vessel in the antegrade direction.

Another factor that physicians might consider when determining whether to insert an introducer sheath into a patient's vessel in the contralateral retrograde manner is the transmittal of force and torque that the physician applies to the procedure equipment. Inserting the procedure equipment in the retrograde direction near the patient's right hip and feeding the equipment through the vessels and then in the antegrade direction in the left side of the patient's body can be problematic because the force and torque that the physician applies to the procedure equipment near the patient's right hip is not transmitted directly to the patient's left ankle. Because the force and torque must change direction as they are transferred within the patient's vessel and are consequently applied at the patient's left ankle, the movement and manipulation of procedure equipment is less accurate, less precise and less successful. Accordingly, the potential for limited manipulation of the procedure equipment at the site of the procedure is another factor that tends to cause a physician to prefer performing a procedure in a patient's vessel in the antegrade direction.

There is a need, therefore, for an improved introducer method and procedural apparatus that avoids the disadvantages of contralateral retrograde access, while also avoiding the problems associated with antegrade entry and exit of vessels.

### SUMMARY

At least some of the embodiments of the present invention address the above-described need by providing a medical procedure access kit including an introducer sheath for inserting a medical device into a biological tubular structure. The kit includes at least one semi-flexible sheath and at least one semi-flexible angled guidewire. The semi-flexible sheath includes a longitudinal opening, a pre-formed bend along a length of the sheath, and a side hole disposed on the pre-formed bend. The semi-flexible angled guidewire is sized and configured so that it can be received within the longitudinal opening of the sheath and fed through the side hole into the biological tubular structure. The sheath and the angled guidewire facilitate entering a biological tubular structure in a retrograde direction and subsequently performing work within the biological tubular structure in an antegrade direction. Further, the sheath and the angled guidewire facilitate performing work within the biological tubular structure in the antegrade direction and then exiting the biological tubular structure in the retrograde direction.

One embodiment of the disclosure provides a method of inserting a medical device into a biological tubular structure. The method includes inserting a sheath over a guidewire at an entry site into a biological tubular structure in a retrograde direction, removing the guidewire from the sheath, threading an angled guidewire through a side hole in the first sheath and into the biological tubular structure in an antegrade direction, removing the sheath from the biological tubular structure, and inserting a second sheath over the angled guidewire into the biological tubular structure in the antegrade direction.

Another embodiment of the disclosure provides a method of removing a medical device from a biological tubular structure. The method includes inserting a guidewire into a sheath at an entry site in the biological tubular structure in an antegrade direction, removing the sheath from the biological tubular structure, inserting a second sheath which has a side hole over the guidewire into the biological tubular structure in the antegrade direction, removing the guidewire from the second sheath, and threading an angled guidewire through the side hole of the second sheath and into the biological tubular structure in the retrograde direction.

The above described features and advantages, as well as others, will become more readily apparent to those of ordinary skill in the art by reference to the following detailed description and accompanying drawings. While it would be desirable to provide an introducer sheath that provides one or more of these or other advantageous features, the teachings disclosed herein extend to those embodiments which fall within the scope of the appended claims, regardless of whether they accomplish one or more of the above-mentioned advantages.

### BRIEF DESCRIPTION OF THE DRAWINGS

Features of the introducer sheath are apparent to those skilled in the art from the following description with reference to the following drawings.

FIG. 1A depicts a top view of an exemplary medical procedure access kit for inserting a medical device into and/or removing a medical device from a biological tubular structure.

FIG. 1B depicts a side view of an introducer sheath of the medical procedure access kit of FIG 1.

FIGS. 2A-2L depict steps of a method of using the medical procedure access kit of FIG. 1 to insert a medical device into a biological tubular structure.

FIGS. 3A-3L depict steps of a method of using the medical procedure access kit of FIG. 1 to remove a medical device from a biological tubular structure.

### DESCRIPTION

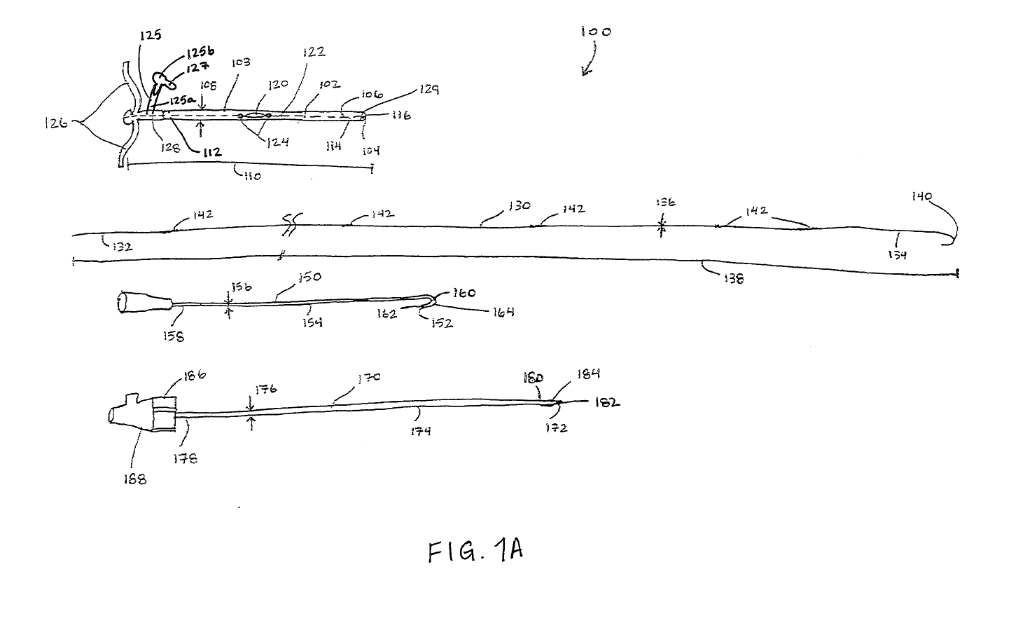

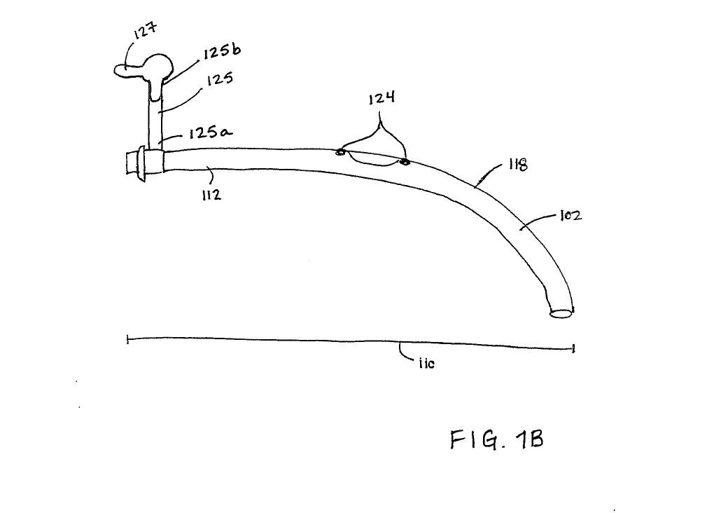

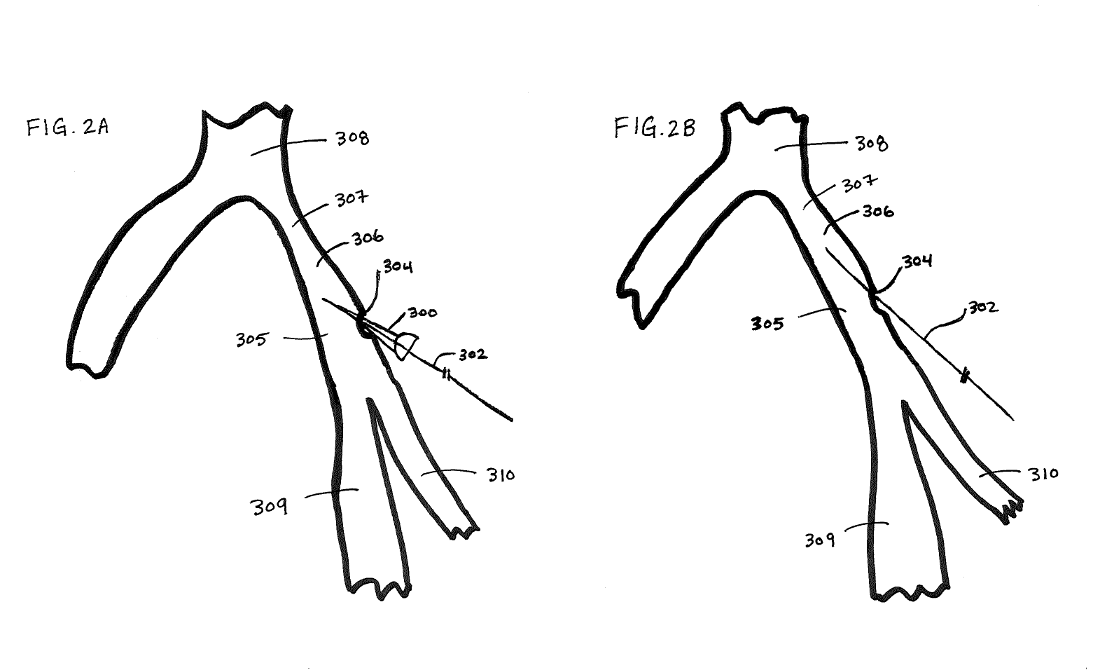

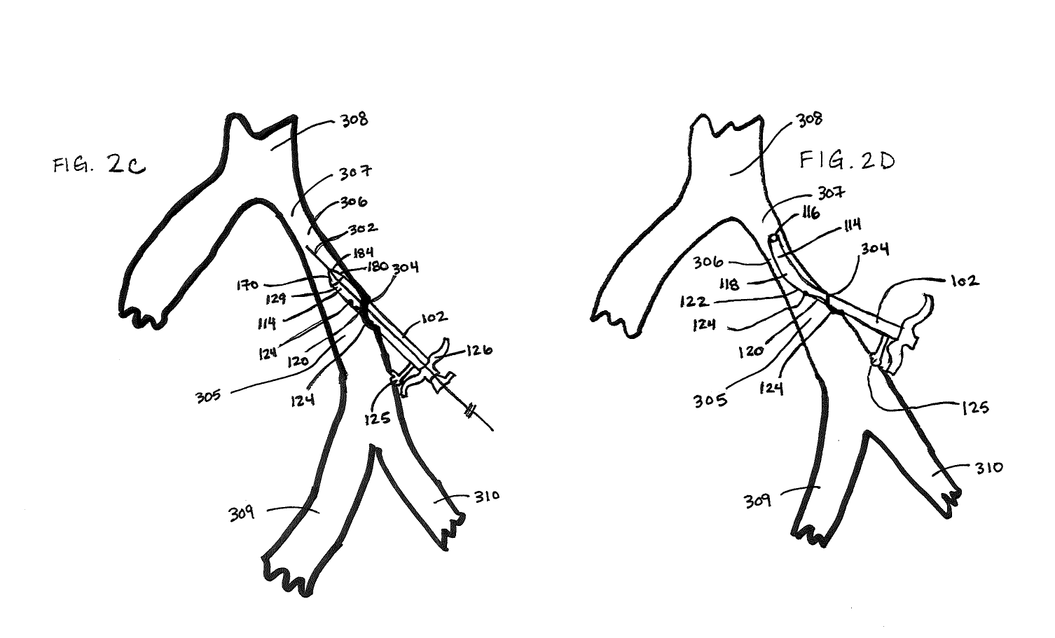

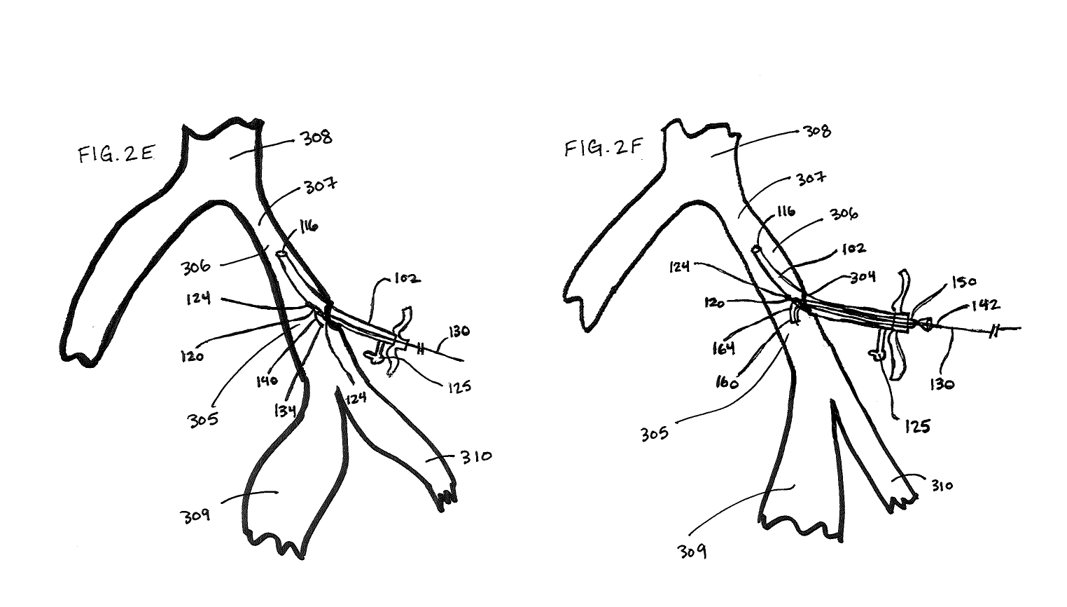

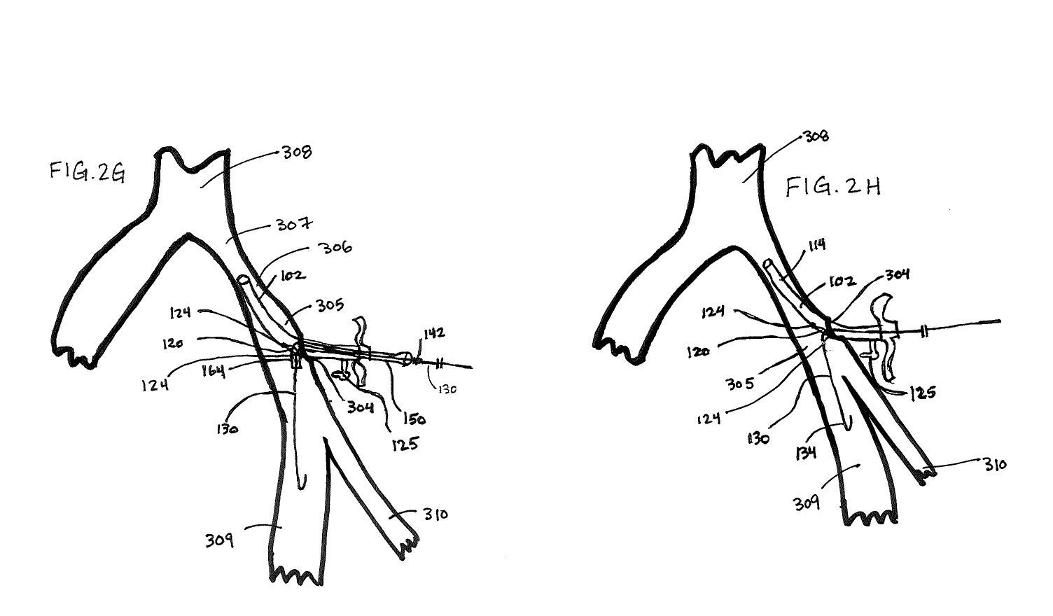

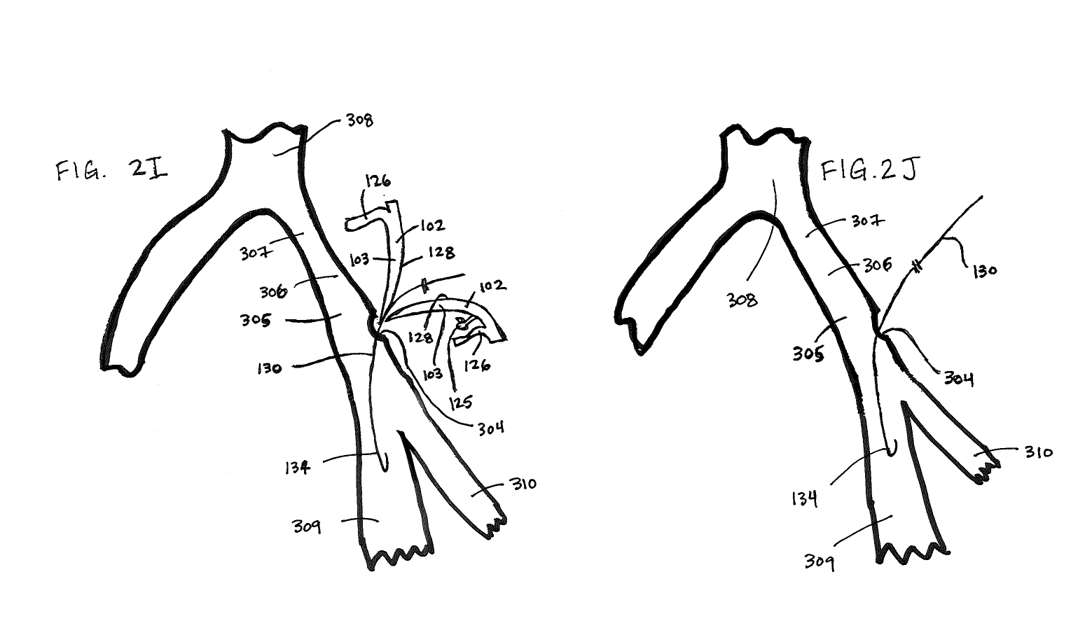

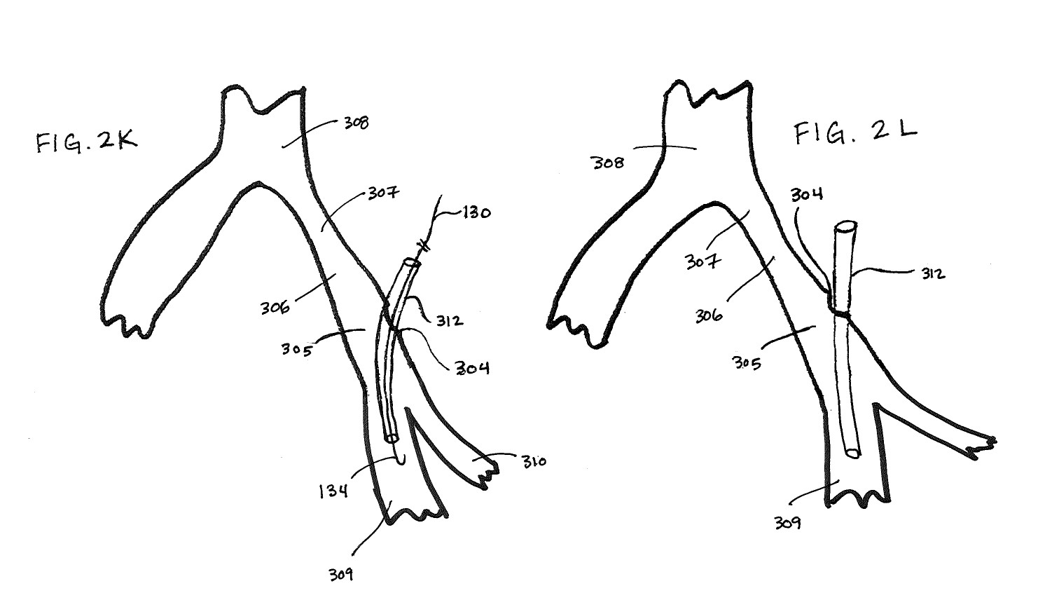

FIG. 1A depicts the elements of a medical procedure access kit 100 for inserting a medical device into and/or removing a medical device from a biological tubular structure, for example, a blood vessel or a bronchial tube. The medical procedure access kit 100 depicted in FIG. 1A is an exemplary embodiment of an access kit including elements to facilitate inserting an endovascular device into a vessel. This kit 100 in its basic form includes an introducer sheath 102, an angled guidewire 130, a j-tip catheter 150, and a dilator 170. In embodiments described herein, the elements included in the access kit 100 can be used in combination with other standard procedure equipment also used for inserting an endovascular device into a vessel. For example, the access kit 100 can be used with a needle 300 (shown in FIG. 2A) and a guidewire 302 (shown in FIG. 2A) to facilitate introducing the introducer sheath 102 using the Seldinger Technique (described below). The access kit 100 can also be used in conjunction with other standard endovascular equipment, such as that associated with various endovascular procedures performed in the antegrade direction.

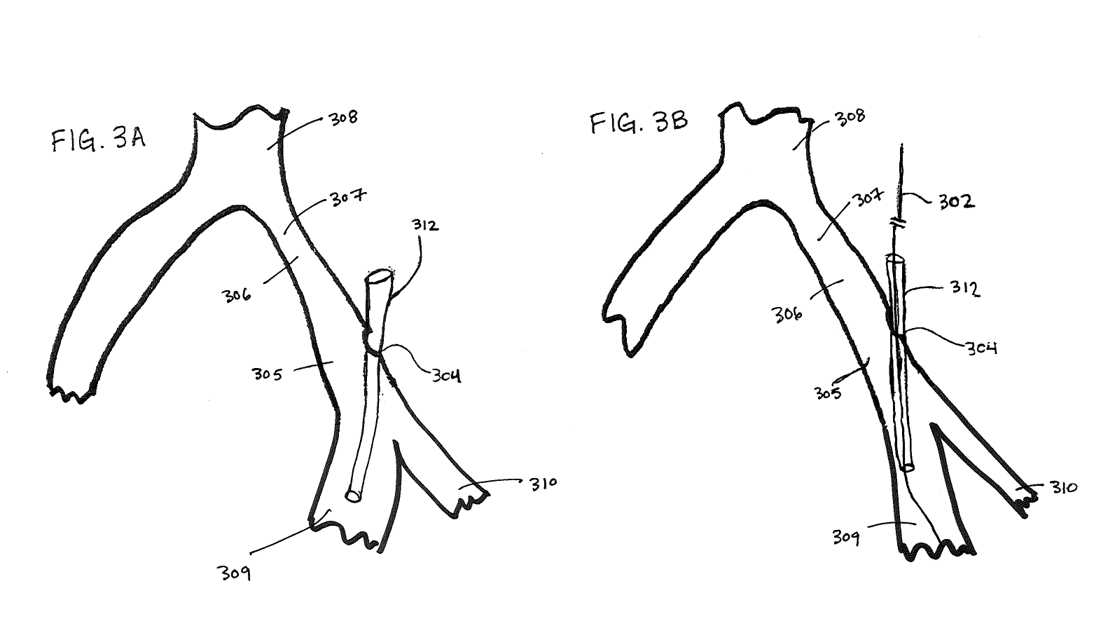

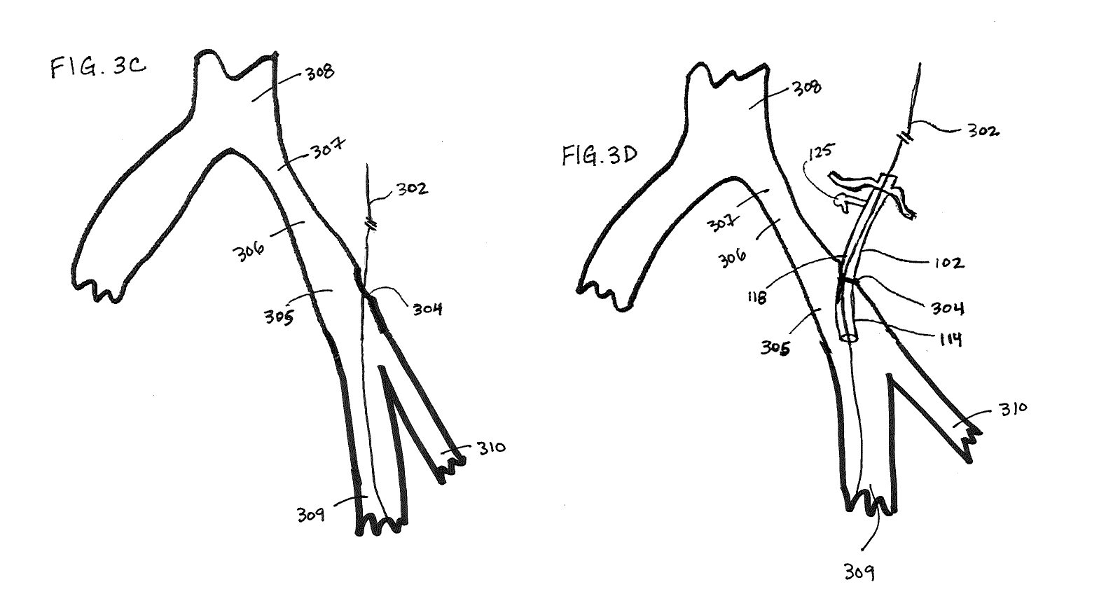

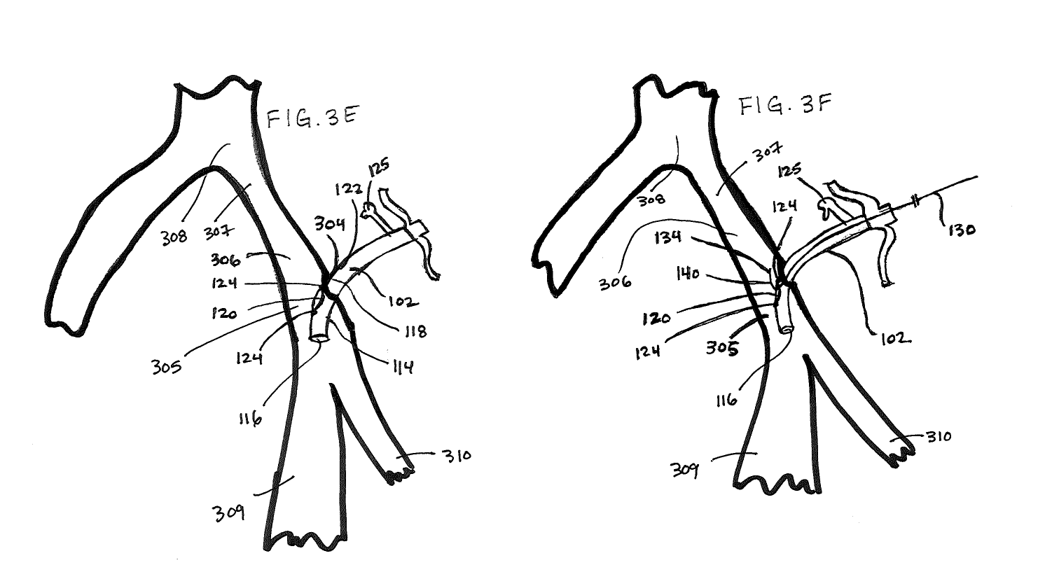

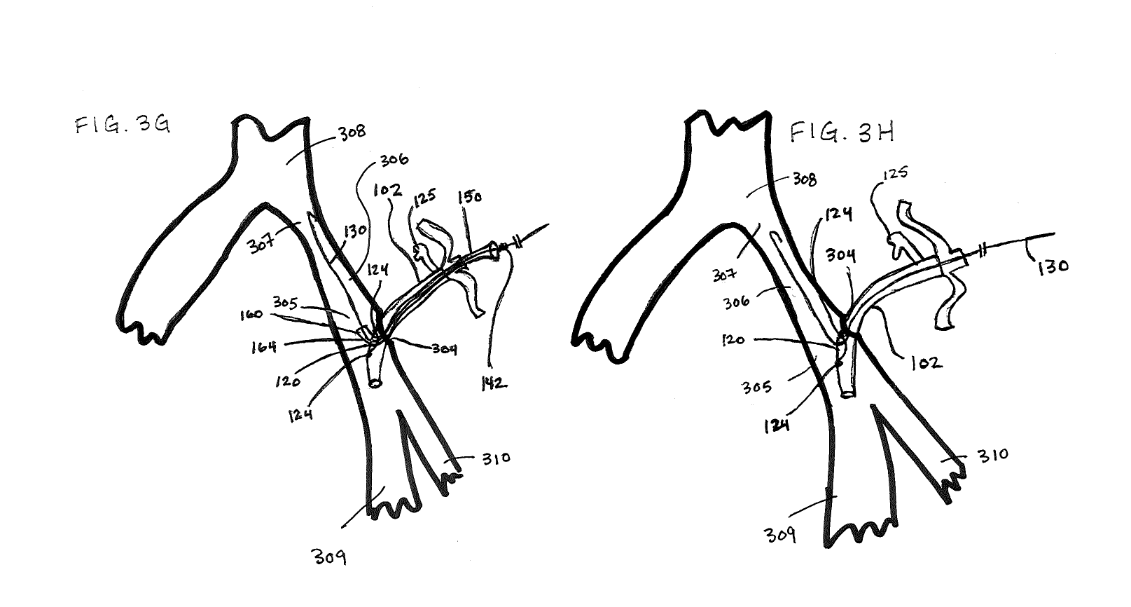

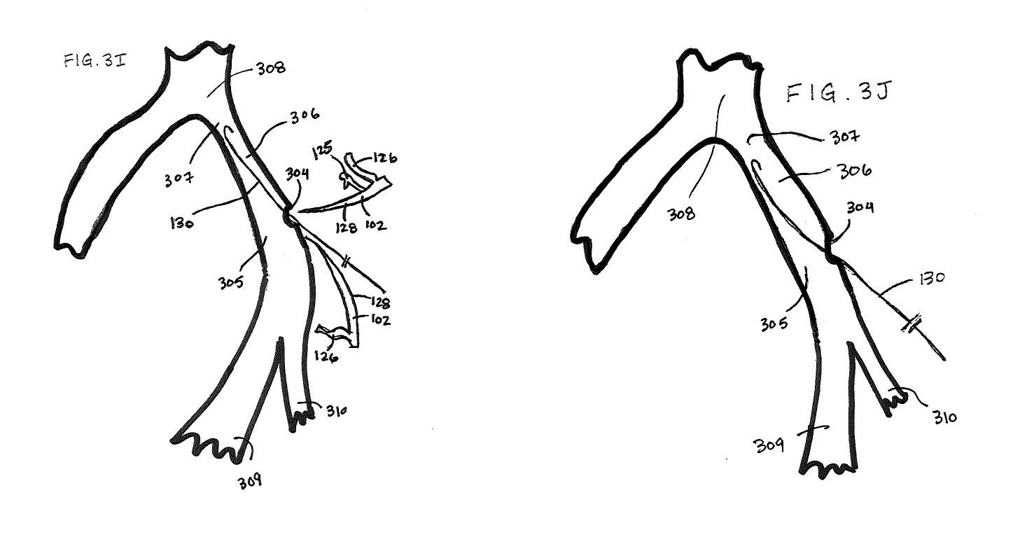

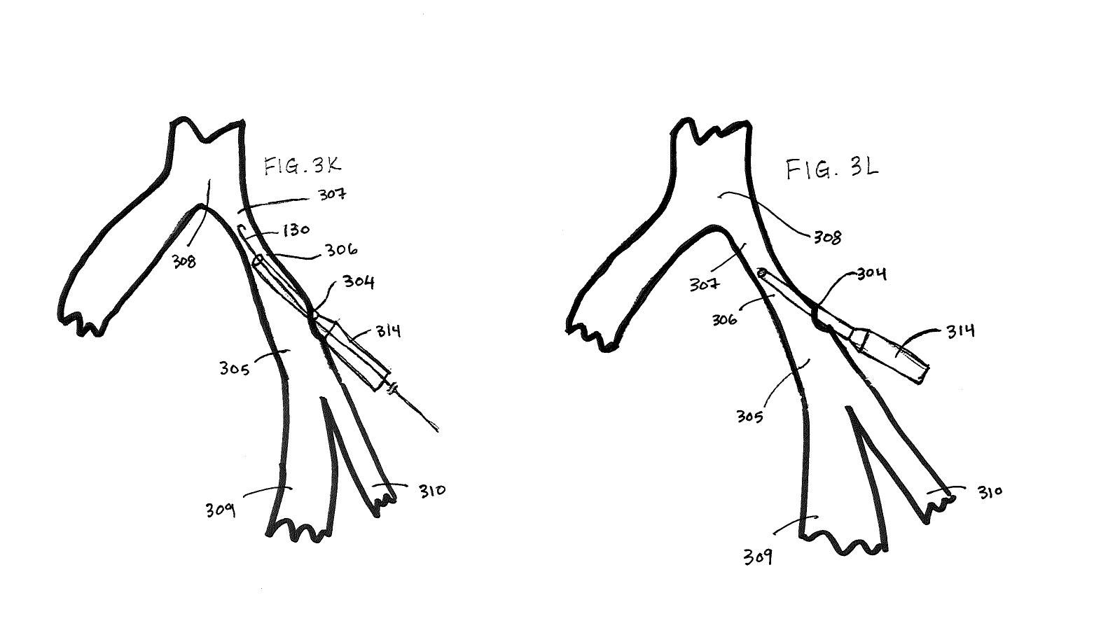

Alternatively, or in addition, the elements of the access kit 100 can be used to facilitate removing an endovascular device from a vessel. To this end, the access kit 100 can be used with a guidewire 302 (shown in FIG. 3B) and a standard or known vascular closing device 310 (shown in FIG. 3K) to facilitate such removal processes. The access kit 100 can also be used in conjunction with standard endovascular equipment. In such a case, the access kit 100 enables a physician to perform endovascular work in the antegrade direction and then exit a vessel in the retrograde direction.

In yet another alternative embodiment, the access kit 100 can include two introducer sheathes 102, two j-tip catheters 150, two angled guidewires 130, and two dilators 170. Such a kit enables a physician to perform both procedures mentioned above including: entering the vessel in the retrograde direction, using one of the introducer sheathes 102, j-tip catheters 150 and angled guidewires 130 to allow the introduction of standard endovascular devices an antegrade direction; and after completion of the endovascular procedure in the antegrade direction, exiting the vessel in the retrograde direction using the second of the introducer sheathes 102, j-tip catheters 150 and angled guidewires 130.

As mentioned above, different standard endovascular equipment can be used with the access kit 100 to facilitate different procedures. For example, the access kit 100 can be used with syringes, standard catheters, balloon catheters, stents, and/or other flexible tubes like work sheath 312 (shown in FIG. 2K) used to perform vascular procedures. In some cases, the standard endovascular equipment mentioned above can also be included in a larger “kit” that includes one or more of the access kits 100.

Referring specifically to FIG. 1A, the introducer sheath 102 includes a tubular member 103 defining an internal side wall 104 and an external side wall 106, a diameter 108, and a length 110. The diameter 108 of the introducer sheath 102 can be, for example, between 4 and 8 Frenches, depending on the size of the medical equipment to be used in the subsequent procedure. The length 110 of the introducer sheath 102 can be, for example, between 10 and 100 centimeters. The introducer sheath 102 includes a proximal end 112, a distal end 114, and a longitudinal opening 116 that is defined by the internal side wall 104.

The introducer sheath 102 is made of a semi-flexible material which enables it to bend relatively easily under applied force, but also enables it to return to its original shape once the applied force is removed. As shown in FIG. 1B, the introducer sheath 102 includes a pre-formed bend 118 along its length 110. When a force is applied to the introducer sheath 102 which causes it to straighten out along its length 110, subsequent removal of that applied force results in the return of the pre-formed bend 118. Accordingly, for example, when the dilator 170 (shown in FIG. 1A) is inserted into the longitudinal opening 116 of the introducer sheath 102, the introducer sheath 102 straightens out along its length 110. Removal of the dilator 170 results in the return of the pre-formed bend 118.

Returning now to FIG. 1A, the introducer sheath 102 includes a side hole 120 extending from the internal side wall 104 to the external side wall 106. Accordingly, the side hole 120 provides an opening between the outside of the introducer sheath 102 and the interior longitudinal opening 116. The side hole 120 is oriented along the convex surface 122 of the pre-formed bend 118 (shown in FIG. 1B) such that it is aimed outwardly away from the introducer sheath 102. In the embodiment shown, the side hole 120 is positioned near an apex of the pre-formed bend 118. In alternative embodiments, however, the side hole 120 may be positioned at other locations along the pre-formed bend 118 such that the side hole 120 is aimed outwardly away from the introducer sheath 102.

The introducer sheath 102 also includes radiopaque markers 124 indicating the location of the side hole 120. The radiopaque markers 124 appear opaque when the introducer sheath 102 is viewed using radiography. The radiopaque markers 124 facilitate identification of the position of the side hole 120 on the introducer sheath 102 during procedure, which aids in the proper positioning of the introducer sheath 102 within the vessel. As shown in FIG. 1A, the radiopaque markers 124 can be provided in the form of dots located on either side of the side hole 120. It is understood, however, that the radiopaque markers 124 can alternatively be provided in any configuration and orientation that effectively indicates the location and position of the side hole 120 when viewed using radiography.

As shown most clearly in FIG. 1B, the introducer sheath 102 also includes a flush port tube 125 having a first end 125a extending from the introducer sheath 102 at a position located near the proximal end 112. The flush port tube 125 also includes a second end 125b at which is located a stopcock 127. The flush port tube 125 is in fluid communication with the interior longitudinal opening 116. The stopcock 127 can be any suitable multi-way stopcock 127 that rotates between “open” and “closed” positions to selectively allow access to (and from) the interior longitudinal opening 116 of the introducer sheath 102 via the flush port tube 125. In accordance with the embodiment described herein, the first end 125a of the flush port tube 125 is arranged on the introducer sheath 102 on a circumferential position that is annularly aligned with the side hole 120. In other words, the flush port tube 125 extends from the introducer sheath 102 in the same radial direction as that which the side hole 120 faces. This arrangement uses an otherwise traditional flush port apparatus to provide a visual indicator to the physician of the rotational orientation of the side hole 120 when the side hole 120 is within the patient's vessel. Specifically, the physician can see how the flush port tube 125 is oriented outside the patient's body to identify how the side hole 120 is oriented within the patient's body. Such visual indication can be used in addition to the radiopaque markers 124, as will be discussed further below in detail.

Returning to FIG. 1A, the introducer sheath 102 also includes grips 126 extending outwardly from the proximal end 112 perpendicularly to the length 110 of the introducer sheath 102. The grips 126 provide ergonomic areas for a physician's fingers to grasp the introducer sheath 102 at the proximal end 112. The introducer sheath 102 also includes weakened structures 128 formed in the grips 126 and continuing along the length 110 of the tubular member 103 to the distal end 114. The weakened structures 128 are configured to facilitate separation of the tubular member 103. Specifically, if the grips 126 are pulled away from one another, the weakened structures 128 allow the tubular member 103 to tear along the length of the introducer sheath 102, thereby separating the introducer sheath 102 into two separate parts. The weakened structures 128 facilitate the introducer sheath 102 cleanly separating into separate parts for easy removal from a patient's vessel. The weakened structures 128 may suitably be thinner wall portions or perforated sections of the tubular member 103 and the grips 126.

The introducer sheath 102 also includes a slight taper 129 formed at the distal end 114 such that the distal end 114 is narrower than the proximal end 112. The taper 129 is wide enough to accommodate the longitudinal opening 116 within the introducer sheath 102, but is narrow enough so that it can assist in separating the tissue, dilating an opening and facilitating entry of the introducer sheath 102 into a vessel.

The angled guidewire 130 includes a proximal end 132 and a distal end 134. The angled guidewire 130 also defines a diameter 136 and a length 138. The angled guidewire 130 is sized and configured to be received within the longitudinal opening 116 of the introducer sheath 102. Accordingly, the diameter 136 of the angled guidewire 130 is smaller than the diameter 108 of the introducer sheath 102. The diameter 136 of the angled guidewire 130 can be, for example, 0.035 inches. The angled guidewire 130 is made of a semi-flexible material which enables it to bend relatively easily under applied force, but also enables it to return to its original shape once the applied force is removed.

The angled guidewire 130 includes a shaped curve 140 formed at the distal end 134. The curve 140 curves back on itself between approximately 70° and 180°. It will be appreciated that any suitable curve shape that curves back on itself between approximately 70° and 180° may be employed. For example, the curve 140 can be approximately “J” shaped, although it is not limited to this particular shape. In any event, the curve 140 can be straightened out through the application of force. Removal of the force, however, results in a return of the curve 140 to the distal end 134 of the angled guidewire 130. In the embodiment described herein, the angled guidewire 130 further includes radiopaque markers 142 spaced at intervals along the length 138 thereof.

The j-tip catheter 150 is a tubular structure that includes an internal side wall 152, an external side wall 154, and a diameter 156. The j-tip catheter 150 includes a proximal end 158, a distal end 160, and a longitudinal opening 162 that is defined by the internal side wall 152. The j-tip catheter 150 is sized and configured to be received within the longitudinal opening 116 of the introducer sheath 102. The j-tip catheter 150 is configured to slide within the longitudinal opening 116 of the introducer sheath 102 with minimal clearance between the external side wall 154 of the j-tip catheter 150 and the internal side wall 104 of the introducer sheath 102. Thus, the diameter 156 of the j-tip catheter 150 is smaller than the diameter 108 of the introducer sheath 102. Additionally, the j-tip catheter 150 is sized and configured to receive the angled guidewire 130 within the longitudinal opening 162 such that the angled guidewire 130 can slide freely through the j-tip catheter 150. Accordingly, the longitudinal opening 162 of the j-tip catheter 150 is larger than the diameter 136 of the angled guidewire 130.

The j-tip catheter 150 is made of a semi-flexible material which enables it to bend relatively easily under applied force, but also enables it to return to its original shape once the applied force is removed. The j-tip catheter 150 includes a shaped curve 164 formed at the distal end 160. Again, the shape of the curve 164 need not be strictly “J” shaped, but can be any shape which curves back on itself at least approximately 70° to 180°. The curve 164 can be straightened out through the application of force. Removal of the force, however, results in a return of the shaped curve 164 to the distal end 160 of the j-tip catheter 150.

Inserting a typical guidewire, like guidewire 302 (shown in FIG. 2A), through the longitudinal opening 162 of the j-tip catheter 150 results in the guidewire 302 originally following the curve 164 of the j-tip catheter 150 upon reaching the distal end 160. Feeding the guidewire further through the j-tip catheter 150, however, causes the curve 164 to straighten out. Removal of the guidewire from the j-tip catheter 150 results in a return of the curve 164 to the distal end 160 thereof.

The dilator 170 defines an internal side wall 172, an external side wall 174, and a diameter 176. The dilator 170 includes a proximal end 178, a distal end 180, and a longitudinal opening 182 that is defined by the internal side wall 172. The dilator 170 is sized and configured to be received within the longitudinal opening 116 of the introducer sheath 102 such that the distal end 180 of the dilator 170 extends just beyond the distal end 114 of the introducer sheath 102. The dilator 170 is configured to slide within the longitudinal opening 116 of the introducer sheath 102 with minimal clearance between the external side wall 174 of the dilator and the internal side wall 104 of the introducer sheath 102. Thus, the diameter 176 of the dilator 170 is smaller than the diameter 108 of the introducer sheath 102. Additionally, the dilator 170 is sized and configured to receive the angled guidewire 130 within the longitudinal opening 182 such that the angled guidewire 130 can slide freely through the dilator 170. Accordingly, the longitudinal opening 182 of the dilator 170 is larger than the diameter 136 of the angled guidewire 130.

The dilator 170 is made of a semi-flexible material which enables it to bend under applied force, but also enables it to return to its original shape once the applied force is removed. The dilator 170 is made of a material which is less flexible than the introducer sheath 102. Suitable materials for the introducer sheath 102 and the dilator 170 having these characteristics are known. The dilator 170 includes a taper 184 formed at the distal end 180 such that the distal end 180 is narrower than the proximal end 178. The taper 184 is wide enough to accommodate the longitudinal opening 182 within the dilator 170, but is narrow enough so that it can assist in spreading tissue apart, dilating an opening and facilitating entry of other endovascular devices into a vessel. The taper 184 is shaped such that there is a relatively smooth transition from the external side wall 174 of the dilator 170 to the external side wall 106 of the introducer sheath 102 when the dilator 170 is inserted into the longitudinal opening 116 of the introducer sheath 102.

The dilator 170 includes a hub 186 and a grip 188 extending from the proximal end 178 of the dilator 170. The grip 188 is connected directly to the dilator 170 and the hub 186 is connected to the grip 188 such that it can rotate around the proximal end 178 of the dilator 170. The hub 186 includes connector threads (not shown) formed on an inside surface that is spaced apart from the dilator 170. The connector threads are configured to engage the grips 126 at the proximal end 112 of the introducer sheath 102 when the introducer sheath 102 is inserted into the dilator 170. Thus, when the dilator 170 is inserted into the introducer sheath 102, rotating the grip 188 relative to the hub 186 causes the dilator 170 to rotate relative to the introducer sheath 102.

As mentioned above, while FIG. 1A shows one introducer sheath 102, one angled guidewire 130, one j-tip catheter 150, and one dilator 170, it is understood that a medical procedure access kit 100 can include other elements in addition to those shown, such as, for example, a needle 300 (shown in FIG. 2A), a guidewire 302 (shown in FIG. 2A), and a work sheath 312 (shown in FIG. 2K). Additionally, a medical procedure access kit 100 may include more than one of each of the elements shown in FIG. 1A. For example, a medical procedure access kit 100 may include two introducer sheaths 102 and two angled guidewires 130. Additionally, a medical procedure access kit 100 may include more than one of each of the elements shown in FIG. 1A wherein the elements have differing dimensions or shapes to be used in circumstances requiring slightly different sized and shaped elements.

By way of example, if a physician uses the medical procedure access kit 100 to enter a patient's vessel in the retrograde direction, perform an endovascular procedure in the antegrade direction, and then exit the patient's vessel in the retrograde direction, then the medical procedure access kit 100 would include at least two introducer sheaths 102. The first sheath 102 would be used to facilitate entering the vessel, as described below with reference to FIGS. 2A-2L, and a second sheath 102 would be used facilitate removal of endovascular surgical equipment from the vessel, as described below with reference to FIGS. 3A-3L.

Referring now to FIGS. 2A-2L, shown is an exemplary method of using the elements described above in a medical procedure access kit 100 to introduce an endovascular device into a patient's femoral artery. As shown in FIG. 2A, the basic anatomy surrounding the femoral artery includes the CFA 305 which extends anteriorly as the EIA 306, which extends anteriorly as the CIA 307, which extends anteriorly as the aorta 308. Posteriorly, the CFA 305 divides into the SFA 309 and the profunda femoris artery (hereinafter “PFA”) 310. In this example, the physician to perform an endovascular procedure in the left SFA 309 accesses the left CFA 305 ipsilaterally in the retrograde direction, and subsequently performs the endovascular procedure in the left SFA 309 in the antegrade direction. It will be understood, however, that the elements of the medical procedure access kit 100 and the steps of the method of using the elements of the medical procedure access kit 100 can be applied to other patient vessels.

As shown in FIG. 2A, introducing an endovascular device into a patient's left CFA 305 using the elements in the medical procedure access kit 100 begins with introducing a needle 300 with a guidewire 302 inserted through the needle 300 into an entry site 304 in the left CFA 305. The needle 300 and the guidewire 302 are inserted into the left CFA 305 in the retrograde direction against the direction of blood flow in the artery. As shown in FIG. 2B, the needle 300 (shown in FIG. 2A) is then removed from the left CFA 305 through the entry site 304 while the guidewire 302 remains inside the left CFA 305. The steps shown in FIGS. 2A and 2B are used to obtain safe access to the vessel and to prepare the vessel for insertion of a sheath or catheter. This technique is commonly referred to as the Seldinger Technique.

Next, as shown in FIG. 2C, the introducer sheath 102 and the dilator 170 are introduced through the entry site 304 over the guidewire 302. The introducer sheath 102 and the dilator 170 follow the guidewire 302 and are also inserted into the left CFA 305 in the retrograde direction. The introducer sheath 102 and the dilator 170 are coupled together with the connector threads of the dilator 170 connected to the grips 126 of the introducer sheath 102. The taper 184 on the distal end 180 of the dilator 170 facilitates spreading tissue apart as the dilator 170 is inserted into the left CFA 305 in order to provide a sufficient opening to receive the taper 129 on the introducer sheath 102. As the introducer sheath 102 and the dilator 170 are further advanced, the taper 129 on the distal end 114 of the introducer sheath 102 further facilitates spreading tissue apart and allowing the tubular member 103 to pass through the entry site 304 and into the left CFA 305. The relative rigidity of the dilator 170 causes the introducer sheath 102 to be substantially straight when entering the left CFA 305 at the entry site 304.

As shown in FIG. 2D, once the introducer sheath 102 is within the CFA 305, the dilator 170 (shown in FIG. 2C) is uncoupled from the introducer sheath 102. Thereafter, the dilator 170 and the guidewire 302 (shown in FIG. 2C) are removed from the CFA 305 through the entry site 304, leaving the distal end 114 of the introducer sheath 102 within the left CFA 305. Removing the dilator 170 from within the introducer sheath 102 allows the introducer sheath 102 to regain its pre-formed bend 118.

Thereafter, the introducer sheath 102 is manipulated such that the side hole 120 faces at least slightly in the antegrade direction and/or in lateral direction facing away from the insertion site 304. To this end, using the position of the flush port tube 125 to identify the orientation of the side hole 120 on the introducer sheath 102, the physician manipulates the introducer sheath 102 to place the side hole 120 such that it faces in the antegrade direction and/or facing laterally away from the insertion site 304. Then, using radiography to distinguish between different materials in the patient's body, the physician views the radiopaque markers 124 on the introducer sheath 102 and more precisely positions the side hole 120 just within the entry site 304. It will be appreciated that in such position, the convex surface 122 of the pre-formed bend 118 allows the side hole 120 to face at least slightly in the antegrade direction as shown in FIG. 2D.

As shown in FIG. 2E, the angled guidewire 130 is then fed through longitudinal opening 116 of the introducer sheath 102. The curve 140 of the angled guidewire 130 is originally deformed so that it will fit within the longitudinal opening 116 of the introducer sheath 102. Once the angled guidewire 130 has been fed through the introducer sheath 102 far enough so that the curve 140 reaches the side hole 120, the angled guidewire 130 is manipulated so that the distal end 134 of the angled guidewire 130 exits the introducer sheath 102 through the side hole 120. As the distal end 134 of the angled guidewire 130 exits the side hole 120, the natural shape of the curve 140 returns. As a result, the distal end 134 of the angled guidewire 130 faces in the antegrade direction within the left CFA 305, as shown in FIG. 2E. Preferably, the angled guidewire 130 is not advanced farther through the side hole 120 than is required to aim the distal end 134 in the antegrade direction.

As shown in FIG. 2F, the j-tip catheter 150 is then inserted over the angled guidewire 130 and fed through the longitudinal opening 116 of the introducer sheath 102. The curve 164 of the j-tip catheter 150 is originally deformed so that it will fit through the longitudinal opening 116 of the introducer sheath 102. Once the j-tip catheter 150 has been fed through the introducer sheath 102 far enough so that the curve 164 reaches the side hole 120, the j-tip catheter 150 is manipulated so that the distal end 160 of the j-tip catheter 150 exits the introducer sheath 102 through the side hole 120. As the distal end 160 of the j-tip catheter 150 exits the side hole 120, the natural shape of the curve 164 returns. As a result, the distal end 160 of the j-tip catheter 150 also faces in the antegrade direction within the left CFA 305.

Once the j-tip catheter 150 is in position, the angled guidewire 130 can be advanced into the left SFA 309 in the antegrade direction. As shown in FIG. 2G, the curve 164 of the j-tip catheter 150 helps guide the angled guidewire 130 in the antegrade direction as the angled guidewire 130 is fed further through the introducer sheath 102 and into the left SFA 309. Using radiography to distinguish between different materials in the patient's body, the physician views the radiopaque markers 142 on the angled guidewire 130 to feed the angled guidewire 130 the correct distance into the left SFA 309. Once the angled guidewire 130 has reached the correct position within the left SFA 309, as shown in FIG. 2H, the j-tip catheter 150 (shown in FIG. 2G) can be removed from the left CFA 305, leaving the distal end 114 of the introducer sheath 102 extending within the CFA 305 and the distal end 134 of the angled guidewire 130 extending within the SFA 309.

Next, as shown in FIG. 2I, the introducer sheath 102 can be removed from the left CFA 305 leaving the distal end 134 of the angled guidewire 130 within the left SFA 309. The introducer sheath 102 is removed from the left CFA 305 by pulling the grips 126 away from each other so that the introducer sheath 102, including the tubular member 103, peels apart along the weakened structures 128. Once in two pieces, the introducer sheath 102 can be easily removed from the left CFA 305 without disrupting the angled guidewire 130. As shown in FIG. 2J, the distal end 134 of the angled guidewire 130 now remains in the left SFA 309 and is oriented in the antegrade direction. After arriving at the step of the method depicted by FIG. 2J, the physician who inserted surgical elements into the left CFA 305 in the retrograde direction in the steps depicted by FIGS. 2A-2F is now able to begin performing an endovascular surgical procedure in the antegrade direction.

To this end, as shown in FIG. 2K, a work sheath 312 for performing a subsequent endovascular procedure can be inserted into the left SFA 309 at the entry site 304 over the angled guidewire 130 in the antegrade direction. As shown in FIG. 2L, the angled guidewire 130 (shown in FIG. 2K) can then be removed from the left SFA 309 leaving the work sheath 312 within the left SFA 309. The work sheath 312 is now ready to be used for procedures involving other endovascular devices within the left SFA 309. Accordingly, after entering the left CFA 305 in the retrograde direction, the physician is now able to perform endovascular surgical procedures in the left SFA 309 in the antegrade direction.

As described above and shown in FIGS. 2A-2L, the medical procedure access kit 100 facilitates changing direction within the CFA 305 so that the SFA 305 can be entered in the retrograde direction (as shown in FIGS. 2A-2D) and the subsequent endovascular procedures can be performed in the antegrade direction (as shown in FIGS. 2K-2L). Thus, the method described above and the kit 100 of FIG. 1A avoids the prior practice of having to enter a different artery in the retrograde direction and then transmitting medical equipment through the vasculature to reach the left SFA 309 in order to perform an endovascular procedure in the antegrade direction in the left SFA 309.

As discussed above, another aspect of the kit 100 of FIG. 1A is that it can additionally or alternatively be used following an endovascular surgical procedure to facilitate exiting and closing the patient's vessel in the retrograde direction. FIGS. 3A-3L depict steps of a method of using the elements described above in a medical procedure access kit 100 to remove an endovascular device from a patient's vessel. For continued clarity of illustration, the steps are described with reference to the femoral artery. In this example, after performing an endovascular procedure in the left SFA 309 in the antegrade direction, the physician subsequently exits the left SFA 309 in the retrograde direction. As with the method of FIGS. 2A-2L, however, that the elements of the medical procedure access kit 100 and the steps of the method described below can be applied to other patient vessels.

As shown in FIG. 3A, the method of closing an antegrade vascular site in a retrograde direction described herein presupposes that a work sheath 312 has been inserted into the left SFA 309 ipsilaterally in an antegrade direction. Accordingly, the method of FIGS. 3A-3L may suitably be used to close a site prepared by the method described above in connection with FIGS. 2A-2L. However, the method of FIGS. 3A-3L may also be used for work sheaths inserted in the antegrade direction by other means.

As shown in FIG. 3B, a guidewire 302 is inserted into the left SFA 309 through the work sheath 312 in the antegrade direction. Next, as shown in FIG. 3C, the work sheath 312 (shown in FIG. 3B) is removed from the left SFA 309 over the guidewire 302 leaving the guidewire 302 in the left SFA 309.

As shown in FIG. 3D, the distal end 114 of an introducer sheath 102 is then inserted into the entry site 304 over the guidewire 302 in the antegrade direction. The pre-formed bend 118 in the introducer sheath 102 causes the guidewire 302 to bend slightly as the introducer sheath 102 is fed over the guidewire 302. As shown in FIG. 3E, once the introducer sheath 102 is inside the left CFA 305, the guidewire 302 (shown in FIG. 3D) is removed from the left SFA 309 leaving the distal end 114 of the introducer sheath 102 within the left CFA 305.

Thereafter, the introducer sheath 102 is manipulated to position the side hole 120 relative to the entry site 304. Using the position of the flush port tube 125 to identify the orientation of the side hole 120 on the introducer sheath 102, the physician manipulates the introducer sheath 102 to place the side hole 120 in a generally correct position and orientation, facing in the retrograde direction and/or inward from the entry site. Then, using radiography to distinguish between different materials in the patient's body, the physician views the radiopaque markers 124 on the introducer sheath 102 and positions the side hole 120 just within the entry site 304. Thus, the physician uses the flush port tube 125 and the radiopaque markers 124 to facilitate aligning the side hole 120 on the convex surface 122 of the introducer sheath 102 so that it faces the retrograde direction within the left CFA 305.

For similar reasons as those discussed above in connection with step 2D, the positioning of the side hole 120 is important to subsequent operations with the introducer sheath 102. The convex surface 122 of the pre-formed bend 118 allows the side hole 120 to be positioned in at least a slightly retrograde position.

Next, as shown in FIG. 3F, the angled guidewire 130 is then fed through longitudinal opening 116 of the introducer sheath 102. The curve 140 of the angled guidewire 130 is originally deformed so that it will fit within the longitudinal opening 116 of the introducer sheath 102. Once the angled guidewire 130 has been fed through the introducer sheath 102 far enough so that the curve 140 reaches the side hole 120, the angled guidewire 130 is manipulated so that the distal end 134 of the angled guidewire 130 exits the introducer sheath 102 through the side hole 120. As the distal end 134 of the angled guidewire 130 exits the side hole 120, the natural shape of the curve 140 returns. As a result, the distal end 134 of the angled guidewire 130 faces in the retrograde direction within the left CFA 305, as shown in FIG. 3F. Preferably, the angled guidewire 130 is not advanced farther through the side hole 120 than is required to aim the distal end 134 in the retrograde direction.

As shown in FIG. 3G, the j-tip catheter 150 is then inserted over the angled guidewire 130 and fed through the longitudinal opening 116 of the introducer sheath 102. The curve 164 of the j-tip catheter 150 is originally deformed so that it will fit within the longitudinal opening 116 of the introducer sheath 102. Once the j-tip catheter 150 has been fed through the introducer sheath 102 far enough so that the curve 164 reaches the side hole 120, the j-tip catheter 150 is manipulated so that the distal end 160 of the j-tip catheter 150 exits the introducer sheath 102 through the side hole 120. As the distal end 160 of the j-tip catheter 150 exits the side hole 120, the natural shape of the curve 164 returns. As a result, the distal end 160 of the angled guidewire 150 also faces in the retrograde direction within the left CFA 305 as shown in FIG. 3G.

Once the j-tip catheter 150 is in position, the angled guidewire 130 can be further advanced in the left CFA 305 in the retrograde direction, and typically into the left EIA 306 and the left CIA 307. As shown in FIG. 3G, the curve 164 of the j-tip catheter 150 helps guide the angled guidewire 130 in the retrograde direction as the angled guidewire 130 is fed further through the introducer sheath 102. Using radiography to distinguish between different materials in the patient's body, the physician views the radiopaque markers 142 on the angled guidewire 130 to feed the angled guidewire 130 the correct distance into the left EIA 306 and the left CIA 307, which are located in the retrograde direction of the left SFA 309. Once the angled guidewire 130 has reached the correct position within EIA 306 and/or CIA 307, as shown in FIG. 3H, the j-tip catheter 150 (shown in FIG. 3G) can be removed, leaving the distal end 114 of the introducer sheath 102 and the distal end 134 of the angled guidewire 130 extending within the SFA 309, the EIA (“EIA”) 305, and the CIA (“CIA”) 307.

Next, as shown in FIG. 3I, the introducer sheath 102 can be removed from the CFA 305 leaving the angled guidewire 130 extending within the EIA 306 and CIA 307. The introducer sheath 102 is removed from the CFA 305 by pulling the grips 126 away from each other so that the introducer sheath 102 peels apart along the weakened structures 128. Once in two pieces, the introducer sheath 102 can be easily removed from the CFA 305 without disrupting the angled guidewire 130. As shown in FIG. 3J, the angled guidewire 130 now remains within the EIA 306, CFA 305 and CIA 307 and is oriented in the retrograde direction.

As shown in FIG. 3K, using the angled guidewire 130 extending in the retrograde direction, any suitable retrograde vessel closing device/method can be employed to close the entry site 304 in the left CFA 305. For example, the Angio-Seal™ device and respective method, the Mynx™ device and respective method, the StarClose™ device and respective method, the Vasoseal™ device and respective method, or the Perclose™ device and respective method can be used to close the entry site 304 of the left SFA 309 in the retrograde direction. The retrograde vessel closing device 314 shown in FIG. 3K is a generic representation of any suitable retrograde vessel closing device and can be inserted into the CFA 305 at the entry site 304 over the angled guidewire 130 in the retrograde direction.

As shown in FIG. 3L, the angled guidewire 130 (shown in FIG. 3K) can then be removed from the left CFA 305 leaving the retrograde vessel closing device 314 within the CFA 305. The retrograde vessel closing device 314 is now ready to be used for a retrograde vessel closing method to properly close the entry site 304 in the left CFA 305.

As described above and shown in FIGS. 3A-3L, the introducer sheath 102 facilitates changing direction within the CFA 305 so that an endovascular procedure can be performed in the SFA 309 in the antegrade direction, and the closure procedure can be carried out in the retrograde direction. Thus, the above-described method avoids the drawbacks of closing an antegrade vascular site, particularly in the femoral artery.

The foregoing detailed description of one or more embodiments of the introducer sheath has been presented herein by way of example only and not limitation. It will be recognized that there are advantages to certain individual features and functions described herein that may be obtained without incorporating other features and functions described herein. Moreover, it will be recognized that various alternatives, modifications, variations or improvements of the above-disclosed embodiments and other features and functions, or alternatives thereof, may be desirably combined into many other different embodiments, systems or applications. Presently unforeseen or unanticipated alternatives, modifications, variations or improvements therein may be subsequently made by those skilled in the art which are also intended to be encompassed by the appended claims. Therefore, the spirit and scope of any appended claims should not be limited to the description of the embodiments contained herein.

What is claimed is:

1. A medical procedure access kit for inserting a medical device into a biological tubular structure comprising:
at least one semi-flexible sheath defining an internal side wall and an external side wall, the at least one sheath including:
a longitudinal opening defined by the internal side wall;
a pre-formed bend along a length of the at least one sheath; and
a side hole disposed on the pre-formed bend and extending from the external side wall to the internal side wall; and
at least one semi-flexible angled guidewire sized and configured to be received within the at least one sheath.

2. The medical procedure access kit of claim 1, wherein the at least one semi-flexible sheath further includes one or more weakened structures to facilitate separation of the sheath.

3. The medical procedure access kit of claim 1, wherein the side hole is disposed on a convex surface of the pre-formed bend.

4. The medical procedure access kit of claim 1, further comprising at least one semi-flexible catheter sized and configured to be received within the at least one sheath.

5. The medical procedure access kit of claim 4, wherein the at least one catheter includes a j-tip at a distal end.

6. The medical procedure access kit of claim 5, wherein the at least one sheath further includes at least one radiopaque marker located on the external side wall adjacent the side hole.

7. The medical procedure access kit of claim 6, further comprising at least one semi-flexible dilator sized and configured to be received within the at least one sheath.

8. The medical procedure access kit of claim 7, further comprising a second semi-flexible sheath defining an internal side wall and an external side wall, the second sheath including:
a longitudinal opening defined by the internal side wall;
a pre-formed bend along a length of the at least one sheath; and
a side hole disposed on the pre-formed bend and extending from the external side wall to the internal side wall.

9. The medical procedure access kit of claim 1, further comprising a port disposed on the at least one semi-flexible sheath and aligned with the side hole.

10. A method of inserting a medical device into a biological tubular structure comprising:
inserting a first sheath over a guidewire at an entry site into a biological tubular structure in a retrograde direction;
removing the guidewire from the first sheath;
threading an angled guidewire through a side hole in the first sheath and into the biological tubular structure in an antegrade direction;
removing the first sheath from the biological tubular structure; and
inserting a second sheath over the angled guidewire into the biological tubular structure in the antegrade direction.

11. The method of claim 10, further comprising aligning the side hole in the first sheath such that it is just within the entry site and faces in an antegrade direction to facilitate threading the angled guidewire through the side hole in the first sheath and into the biological tubular structure in the antegrade direction.

12. The method of claim 10, further comprising inserting a j-tip catheter through the side hole, and into the biological tubular structure over the angled guidewire to facilitate feeding the angled guidewire into the biological tubular structure in the antegrade direction.

13. The method of claim 12, further comprising aligning the j-tip catheter just within the biological tubular structure to facilitate feeding the angled guidewire into the biological tubular structure in the antegrade direction.

14. The method of claim 12, further comprising removing the j-tip catheter and the sheath from the biological tubular structure, leaving the angled guidewire in the biological tubular structure at a location downstream from the entry site.

15. The method of claim 10, wherein removing the sheath from the biological tubular structure includes separating the sheath into at least a portion.

16. A method of removing a medical device from a biological tubular structure comprising:
inserting a guidewire into a first sheath at an entry site in the biological tubular structure in an antegrade direction;
removing the first sheath from the biological tubular structure;
inserting a second sheath over the guidewire into the biological tubular structure in the antegrade direction, the second sheath having a side hole;
removing the guidewire from the second sheath; and
threading an angled guidewire through the side hole of the second sheath and into the biological tubular structure in the retrograde direction.

17. The method of claim 16, further comprising aligning the side hole in the second sheath such that it is just within the entry site and faces in a retrograde direction to facilitate threading the angled guidewire through the side hole in the second sheath and into the biological tubular structure in the retrograde direction.

18. The method of claim 16, further comprising inserting a j-tip catheter through the side hole in the second sheath, and into the biological tubular structure over the angled guidewire to facilitate feeding the angled guidewire into the biological tubular structure in the retrograde direction.

19. The method of claim 18, further comprising removing the j-tip catheter and the second sheath from the biological tubular structure, leaving the angled guidewire in the biological tubular structure at a location upstream from the entry site.

20. The method of claim 16, wherein removing the second sheath from the biological tubular structure includes separating the second sheath into at least a portion.

21. The method of claim 16, further comprising closing the biological tubular structure using a retrograde biological tubular structure closing technique.
